(19) United States
(12) Patent Application Publication
(10) Pub. No.: US 2006/0121479 A1
(43) Pub. Date: Jun. 8, 2006

(54) Molecular size markers for species identification of mycobacteriae

(76) Inventors: Orhan Kaya Koksalan, Istanbul (TR); Tanil Kocagoz, Istanbul (TR)

Correspondence Address: ARENT FOX PLLC, 1050 CONNECTICUT AVENUE, N.W., SUITE 400, WASHINGTON, DC, 20036, US

(21) Appl. No.: 10/528,453

(22) PCT Filed: Sep. 26, 2003
(86) PCT No.: PCT/TR03/00082

§ 371 Date: Oct. 11, 2005

(30) Foreign Application Priority Data
Sep. 26, 2002 (TR) 2002/02252

(51) Int. Cl. C12Q 1/68; C12N 9/22; C12N 1/21; C07H 21/04

(52) U.S. Cl. 435/6; 435/199; 435/252.3; 536/24.1

(57) ABSTRACT

This invention consists of DNA molecular size markers which are used to determine in a correct and easy way the size of DNA fragments in the evaluation step of the sizes of DNA restriction fragments separated by electrophoresis, of the hsp65 PCR-REA method used for species identification of mycobacteriae.

### CROSS-REFERENCE TO RELATED APPLICATION

This application is a National Stage entry of International Application No. PCT/TR2003/000082, filed Sep. 26, 2003. The disclosure of the prior application is hereby incorporated herein in its entirety by reference.

### BACKGROUND OF THE INVENTION

Various mycobacterial species like Mycobacterium tuberculosis cause infections in humans. Antibacterial agents that are effective in the treatment of infections caused by different mycobacterial species may vary greatly. For this reason, identification of mycobacteriae recovered from clinical specimens is of prime importance. As most of the mycobacteriae necessitate substantially longer times (3 to 8 weeks) to be grown in cultures, culture-based conventional identification methods like pigment formation, colony morphology and biochemical tests have the disadvantages of being slow and ambiguous. Recently, molecular identification techniques yielding unambiguous results within a few days has been developed. One of these techniques, the so-called hsp65 PRA (Polymerase chain reaction-restriction enzyme analysis) has ever gaining widespread use due to its rapidity, ease and lower cost. This method is based on the amplification of a 441 bp-long fragment of the gene encoding the 65-kDA heat shock protein, digestion of the PCR products by the restriction enzymes BstEII and HaeIII, and separation of the cleaved products by agarose or better by polyacrylamide gel electrophoresis. The sizes of each of the fragments are calculated by comparing them with the bands of a molecular size marker and finally comparing the resulting restriction enzyme pattern with that of known mycobacterial strains given in the algorithm.

The lack of an appropriate molecular size marker with bands exactly the same size as the restriction fragments encountered in hsp65 PCR-REA of mycobacteriae is one of the major shortcomings of this technique for, the lack of such a size marker renders visual interpretation of the restriction fragments' sizes impossible and mandates manual or software-assisted calculations. The subject of this invention is to resolve this problem by creating new molecular size markers containing fragments exactly the same size as the BstEII and HaeIII digested fragments that could have been obtained in hsp65 PCR-REA of mycobacteriae and in this way to enable the correct interpretation of the sizes of restriction fragments separated by electrophoresis by naked eye. These products that are related to the field of molecular microbiology are not directed to diagnose a disease.

Nearly two billion people is estimated to be infected with Mycobacterium tuberculosis which is the most important infectious species within the Mycobacterium genus. Each year about ten million people is contracting the disease and three million die of tuberculosis. Early detection of tuberculosis cases, their proper treatment, and breaking the transmission chain are of paramount importance for effective tuberculosis control. Proper treatment of cases can be achieved by the contribution of rapid, unambigious identification of mycobacteriae up to species level and by determining the susceptibility patterns of the strains.

Despite the fact that Mycobacterium tuberculosis makes up the great majority of mycobacterial infections, there are several other mycobacterial species causing human infections. These bacteriae called “atypical” or “nontuberculous” mycobacteriae show great diversity in their ability to cause disease, they may be

always pathogenic species (Mycobacterium leprae)

potentially pathogenic species (Mycobacterium avium etc.)

not pathogenic ones (Mycobacterium gordonae etc.)

Even the nonpathogenic species may be of clinical importance for, they are capable of living freely in the environment including tap water and thus may contaminate cultures which are used to diagnose the disease.

Identification of mycobacteriae to the species level is important for patient management, as the results obtained influence the decision to treat or not to treat the patient, the choice of proper treatment and the eventual need for patient isolation.

Classical identification methods based on culture and biochemical tests may take weeks to months after reception of specimens and the tests sometimes fail to produce a precise identification. Genotypic methods for the identification of mycobacteriae has been developed in recent years. These molecular methods are of increasing importance because they yield rapid and, mostly, unequivocal results.

Molecular techniques based on probe hybridization or on amplification of specific genomic region provide results limited to a single species per experiment Limited number of commercially available probes that are used in probe hybridization technique restricts the differentiating power of this technique with only a few species, the remaining—approx. 90-mycobacterial species cannot be identified and additionally, each extra probe used to identify the isolate adds an extra cost.

In contrast to the above-mentioned techniques needing specialized equipment, PCR-restriction fragment length polymorphism analysis (PRA) of the hsp65 gene present in all mycobacteriae offers an easy, rapid and inexpensive procedure to identify most of the mycobacterial species in a single experiment. Although hsp65 gene is present in all mycobacteriae, the nucleic acid sequence of hsp65 in each species varies from the others. These variances are made discernible by restriction enzymes that recognize specific DNA sequences and cut the DNA at these sites.

In PRA, a 441 base pair (bp) portion “Telenti fragment” of hsp65 gene is amplified, the amplified products will then be separated into two microcentrifuge tubes and digested by the restriction enzymes BstEII and HaeIII. The digested DNA fragments and a molecular size marker are loaded onto a gel, run in electrophoresis buffer and thus are separated from each other.

The sizes of each of the digested fragments are determined by comparing them with the bands of the molecular size marker whose band sizes are known. In order to achieve more accurate size calculations, this comparison are carried out by using sophisticated computer programs.

The molecular size markers which are the subject of this invention contain fragments exactly the same size as the restriction fragments of mycobacteriae. These molecular size markers specific to PRA of mycobacteriae alleviates the accurate size determination oftested mycobacterial strains' restriction fragments without the need for sophisticated computer programs.

Previously Available Products (Molecular Size Markers)

Electrophoresis is a molecular biological technique that is widely used in determining the sizes of macromolecules. In this technique, electrically charged molecules are run within a cell-forming gel by the application of electrical voltage. During their run through the pores of the gel, molecules are propelling with different velocities depending to the resistance they are facing. For DNA molecules, this velocity is inversely proportional to the logarithm of their sizes. The determination of DNA fragments' sizes separated by electrophoresis is achieved by using molecular size markers that are run in adjacent lanes of the same gel as the DNA fragments of concern and whose fragment sizes are known. For the precise determination of DNA fragments in question, molecular size markers should ideally contain fragments exactly the same size as the tested DNA fragments or at least fragments similar to the sizes of the DNA in concern. This is the reason why commercially available molecular size markers are far from being useful; they do not contain fragments exactly the same size as the tested DNA fragments which renders impossible the easy visual interpretation of digested mycobacterial DNA fragments and separated by electrophoresis; sophisticated software packages are needed to obtain “nearly correct” fragment sizes.

It is common practice to obtain molecular size markers by digestion of bacteriophages having short DNA sequences with restriction enzymes. The most frequently used molecular size marker in published hsp65 PRA studies, is HaeIII-digested φDX174 bacteriophage DNA which is easily reproducible in E. coli. This marker contains eleven fragments which are 1352, 1078, 872, 603, 310, 281, 271, 234, 194, 118, 72 base pairs long. Digesting the 441 bp long fragment of the hsp65 gene either by BstE-1 or HaeIII results in several shorter fragments which mostly range between 50-200 base pairs and are close to each other in size. The size marker φX174/HaeIII has only three bands within this range (194, 118 and 72 bp). Additionally these three bands are not identical to mycobacterial fragments in size. In brief the presence of a few non-identical fragments renders φX174/HaeIII marker unsuitable for PRA.

In comparison, another molecular size marker, φX174/HaeIII, seems to be more appropriate for PRA; it has relatively more bands within the size range of 50-200 bp. However, none of the bands of this marker (726, 713, 553, 500, 427, 417, 413, 311, 249, 200, 151, 140, 118, 100, 82, 66, 48, 42, 40 ve 24 bp) is identical to mycobacterial restriction fragments.

Although the main determinant of the electrophoretic ability of a fragment is its size, the composition of the DNA (GC/AT content) also plays some role. GC/AT content of mycobacteriae is very different from the GC/AT content of bacteriophages. This leads to another erratic calculation of the fragment sizes.

The Purpose of the Invention:

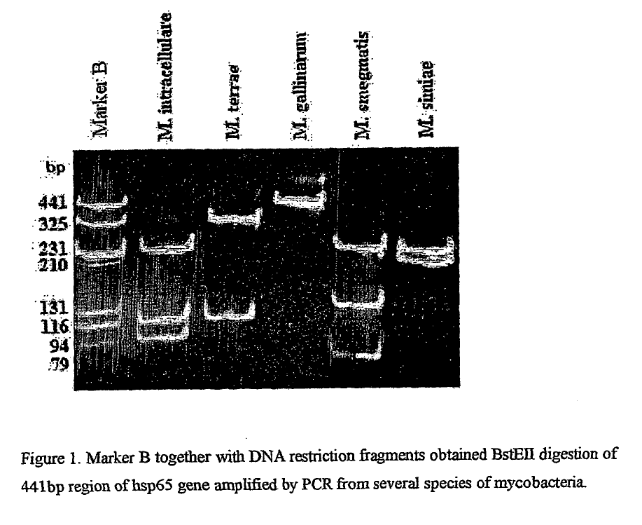

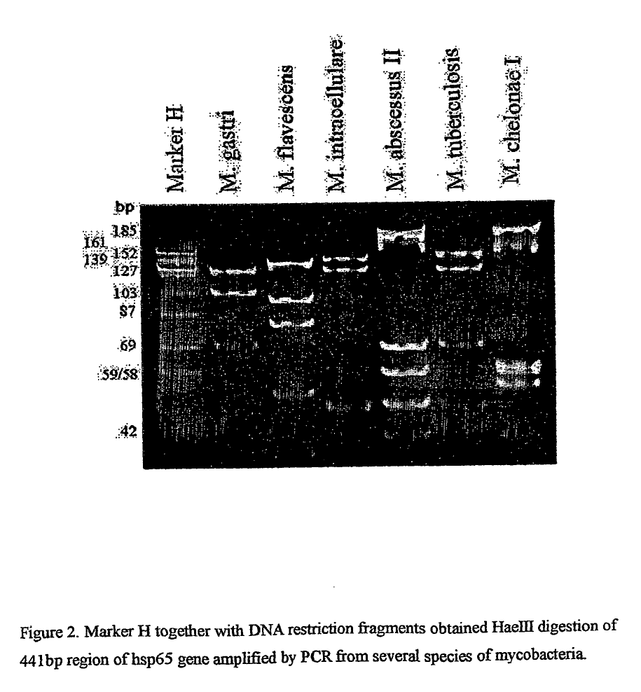

The purpose of this invention is to obtain DNA molecular size markers that will be used in the correct and easy interpretation of the sizes of restriction fragments of 441 bp region of hsp65 gene amplified by PCR and digested by BstEII and HaeIII enzymes, by electrophoretic analysis. For convenience the two molecular size marker are called Marker-B and Marker-H. Marker B contains 8 different size fragments of 441, 325, 231, 210, 131, 116, 94 and 79 bp (FIG. 1). Marker-H contains contains 14 different size fragments of 185, 161, 152, 139, 127, 103, 87, 69, 59, 58, 42, 40, 36 and 34 bp (FIG. 2).

In FIG. 1 Marker B is shown together by restriction fragments obtained by PCR amplification of hsp65 gene of several species of mycobacteriae and digestion by BstEII enzyme and separated by electrophoresis using 6% polyacrilamide gel.

In FIG. 2 Marker H is shown together by restriction fragments obtained by PCR amplification of hsp65 gene of several species of mycobacteriae and digestion by Haem enzyme and separated by electrophoresis using 8% polyacrilamide gel.

### DETAILED DESCRIPTION OF THE INVENTION

For obtaining two molecular size markers standards M. simiae, M. smegmatis, M. gallnarum, M. intracellulare, M. terrae are used to prepare Marker B and M. simiae, M. gallinarum, M. chitae, M. xenopi are used to prepare Marker-H.

Isolation of DNA from Mycobacteriae:

A few mycobacterial colonies from freshly grown cultures are suspended in TE buffer (10 mM Tris pH 8.0, 1 mM EDTA) in plastic microcentrifuge tubes. The suspension is centrifuged at 12000 g for 1 minute to sediment the bacteria and the supernatant is discarded. The bacteria are washed twice by repeating the same process. The final bacterial sediment is suspended in 250 μl TE buffer. The tubes are incubated in a boiling water bath for 20 minutes to lyse the bacteria and release DNA. Cell debris are sedimented by centrifugation and the supernatant which contains mycobacterial DNA is transferred to a clean tube. This DNA is used as template for amplification of hsp65 gene by PCR.

PCR Amplification of hsp65 Gene:

The template DNA obtained from mycobacteriae is mixed in appropriate concentrations of Taq polymerase enzyme, deoxynucleotide triphosphates, enzyme reaction buffer and the primers TB11 (5′ ACC AAC GAT GGT GTG TCC AT 3′) and TB12 (5′ CTT GTC GAA CCG CAT ACC CT 3′). Reaction tubes are placed in a thermal cycler. After denaturation for 5 minutes at 95° C., 40 cycles consisting of 30 seconds at 94° C., for denaturation, 45 seconds at 54° C. for primer annealing and 90 seconds at 72° C. for polymerization is done. Ten minutes at 72° C. is added to the end, to complete possible incomplete strands.

Purification of DNA Amplification Products:

The 441 base pairs (bp) amplified region of mycobacterial hsp65 gene regions are separated in 1% agarose gel by electrophoresis, stained by ethidium bromide and visualized by ultraviolet light. The bands containing these DNA fragments are cut and agarose is solubilized in sodium iodide solution by heating. The released DNA are sedimented by binding to glass milk. After several wash steps DNA is solubilized in sterile deionized water.

Molecular Cloning:

PCR products are cloned into PCR products cloning plasmid vector. For this purpose purified hsp65 gene region are mixed with the plasmid vector and ligated to each other by adding ligase enzyme, enzyme buffer and ATP. The cloned plasmids are introduced into competent E. coli cells. Transformed bacteria are multiplied by culturing and stocks are stored at −85° C.

Plasmid Isolation:

Fresh cultures are prepared from bacterial stocks. Overnight cultures are used for plasmid isolation. Plasmids are purified by alkaline lysis method. For this purpose, bacteria are suspenden in glucose-tris-EDTA buffer and lysed by NaOH-SDS solution Cell walls, proteins and chromosomal DNA are sedimented by potassium acetate. Supernatant that contains plasmid DNA and RNAs is transferred into a clean tube. RNA is eliminated by the addition of RNAse.

Preparation of Molecular Size Markers:

The 441 bp gene region is amplified by PCR, as described above, using hsp65 gene cloned plasmids as template. To prepare Marker-B PCR products obtained from plasmids containing hsp65 gene from M. simiae, M. smegmatis, M. gallinarum, M. intracellulare, M. terrae are mixed with restiction enzyme BstEII, enzyme buffer and incubated 24 hours at 60° C. To prepare Marker-H PCR products obtained from plasmids containing hsp65 gene from M. tuberculosis, M simiae, M. gallinarum, M. chitae, M. xenopi are mixed with restiction enzyme HaeIII, enzyme buffer and incubated 24 hours at 37° C. The products obtained are mixed with gel loading buffer to make them ready to use in electrophoresis. Marker-B obtained this way contains 8 DNA fragments of sizes 441, 325, 231, 210, 131, 116, 94 ve 79 bp (FIG. 1) and Marker-H contains 14 DNA fragments of sizes 185, 161, 152, 139, 127, 103, 87, 69, 59, 58, 42, 40, 36, 34 bp.

1. A DNA molecular size marker comprising DNA fragments of 441, 325, 231, 210, 131, 116, 94 and 79 base pairs.

2. A method for the production of the molecular size marker of claim 1, the method comprising:
a) isolation of DNA from mycobacteriae,
b) amplification of hsp65 gene by PCR,
c) purification of DNA amplification products,
d) molecular cloning into a plasmid vector,
e) isolation of the plasmid vector, and
f) restriction enzyme digestion.

3. The method of claim 2, wherein the species of mycobacteriae used for the isolation of DNA produce DNA fragments of 441, 325, 231, 210, 131, 116, 94 and 79 base pairs.

4. The method of claim 3, wherein the species of mycobacteriae is selected from the group consisting of M. simiae, M. smegmatis, M. gallinarum, M. intracellulare, and M. terrae.

5. The method of claim 2, wherein primers TB11 (5′ ACC MC GAT GGT GTG TCC AT 3′), and TB12 (5′ CTT GTC GM CCG CAT ACC CT 3′) are used in the amplification of hsp65 gene.

6. The method of claim 2, wherein the restriction enzyme is BstEII.

7. A method for determining the size of restriction fragments obtained by BstEII digestion during electrophoretic analysis of hsp65 by PCR-REA, the method comprising the molecular size marker of claim 1.

8. A DNA molecular size marker comprising DNA fragments of 185, 161, 152, 139, 127, 103, 87, 69, 59, 58, 42, 40, 36 and 34 base pairs.

9. A method for the production of the molecular size marker of claim 8, the method comprising:
a) isolation of DNA from mycobacteriae,
b) amplification of hsp65 gene by PCR,
c) purification of DNA amplification products,
d) molecular cloning into a plasmid vector,
e) isolation of the plasmid vector, and
f) restriction enzyme digestion.

10. The method of claim 9, wherein the species of mycobacteriae used for the isolation of DNA produce DNA fragments of 185, 161, 152, 139, 127, 103, 87, 69, 59, 58, 42, 40, 36 and 34 base pairs.

11. The method of claim 10, wherein the species of mycobacteriae is selected from the group consisting of are M. tuberculosis, M. simiae, M. gallinarum, M. chitae, and M. xenopi.

12. The method of claim 9, wherein primers TB11 (5′ ACC MC GAT GGT GTG TCC AT 3′), and TB12 (5′ CTT GTC GM CCG CAT ACC CT 3′) are used in the amplification of hsp65 gene.

13. The method of claim 9, wherein the restriction enzyme is HaeIII.

14. A method for determining the size of restriction fragments obtained by HaeIII digestion during electrophoretic analysis of hsp65 by PCR-REA, the method comprising the molecular size marker of claim 8.
